(19) United States
(12) Patent Application Publication
(10) Pub. No.: US 2022/0051168 A1
(43) Pub. Date: Feb. 17, 2022

(54) INFORMATION PROCESSING APPARATUS, INFORMATION PROCESSING SYSTEM, AND NON-TRANSITORY STORAGE MEDIUM

(71) Applicant: Toyota Jidosha Kabushiki Kaisha, Toyota-shi Aichi-ken (JP)

(72) Inventors: Yurika Tanaka, Yokosuka-shi (JP); Shuichi Sawada, Nagoya-shi (JP); Shin Sakurada, Toyota-shi (JP); Yasuhiro Baba, Gifu-ken (JP); Shintaro Matsutani, Kariya-shi (JP); Tomoya Makino, Kariya-shi (JP)

(21) Appl. No.: 17/398,586

(22) Filed: Aug. 10, 2021

(30) Foreign Application Priority Data
Aug. 12, 2020 (JP) 2020-136167

(51) Int. Cl. G06Q 10/06; G16H 20/70

(52) U.S. Cl. CPC G06Q 10/063116; G16H 20/70; G06Q 10/06398; G06Q 10/06393

(57) ABSTRACT

An information processing apparatus, comprises a controller configured to execute: obtaining scheduled behavior information that is information about a scheduled behavior of a user; obtaining user behavior information that is information about a behavior performed by the user; calculating a deviation degree between the scheduled behavior and the behavior performed by the user, based on the user behavior information and the scheduled behavior information; and performing a predetermined process when the deviation degree exceeds a first value.

### CROSS REFERENCE TO THE RELATED APPLICATION

This application claims the benefit of Japanese Patent Application No. 2020-136167, filed on Aug. 12, 2020, which is hereby incorporated by reference herein in its entirety.

### BACKGROUND

### Technical Field

The present disclosure relates to a technology for assisting users.

### Description of the Related Art

There is a technology for predicting change in physical conditions of users by statistically processing data. For example, Patent Literature 1 discloses a health management system that analyzes the correlation between physical information on a user and an environment condition (e.g., a weather condition) serving as an external factor of change in physical condition, and predicts the change in the user's physical condition.

### CITATION LIST

### Patent Literature

Patent Literature 1: Japanese Patent Laid-Open No. 2013-238970
Patent Literature 2: International Publication No. WO 2017/199663

### SUMMARY

The invention described in Patent Literature 1 can predict the change in physical condition due to an environmental factor.

On the other hand, there is a demand to detect change in the mental condition of a user (for example, deterioration of the mental condition and the like due to stress or the like). However, it is difficult to estimate the change in the human mental condition from the environmental factor.

The present disclosure has an object to provide a technology of estimating deterioration of the mental condition of the user.

The present disclosure in its one aspect provides an information processing apparatus, comprising a controller configured to execute: obtaining scheduled behavior information that is information about a scheduled behavior of a user; obtaining user behavior information that is information about a behavior performed by the user; calculating a deviation degree between the scheduled behavior and the behavior performed by the user, based on the user behavior information and the scheduled behavior information; and performing a predetermined process when the deviation degree exceeds a first value.

The present disclosure in its another aspect provides an information processing system, comprising: a user terminal carried by a user; and a server apparatus, wherein the user terminal includes a first controller configured to periodically transmit position information to the server apparatus, and the server apparatus includes a second controller configured to execute: obtaining scheduled behavior information that is information about a scheduled behavior of the user; generating user behavior information that is information about a behavior performed by the user, based on the position information transmitted from the user terminal; calculating a deviation degree between the scheduled behavior and the behavior performed by the user, based on the user behavior information and the scheduled behavior information; and performing a predetermined process when the deviation degree exceeds a first value.

The present disclosure in its another aspect provides a non-transitory computer readable storing medium recording a computer program for causing a computer to perform an information processing method comprising: obtaining scheduled behavior information that is information about a scheduled behavior of a user; obtaining user behavior information that is information about a behavior performed by the user; calculating a deviation degree between the scheduled behavior and the behavior performed by the user, based on the user behavior information and the scheduled behavior information; and performing a predetermined process when the deviation degree exceeds a first value.

Another aspect may be an information processing method executed by the information processing apparatus (server apparatus) described above, or a computer-readable storage medium that non-transitorily stores the program described above.

According to the present disclosure, the fact that the mental condition of the user deteriorates can be estimated.

### BRIEF DESCRIPTION OF THE DRAWINGS

FIG. 1 is a diagram illustrating an overview of an evaluation system;

FIG. 2 is a diagram illustrating configuration elements of the evaluation system according to a first embodiment in detail;

FIG. 3 is a diagram illustrating processes that a controller executes;

FIG. 4A is a diagram illustrating position information data stored in a storage;

FIG. 4B is a diagram illustrating a behavior model stored in the storage;

FIGS. 5A and 5B are diagrams illustrating the deviation between behaviors;

FIG. 6 is a diagram illustrating a process of calculating a deviation degree;

FIG. 7 is a diagram illustrating a threshold set for the deviation degree;

FIG. 8 is a flowchart of processes that the controller executes in the first embodiment; and

FIG. 9 is a diagram illustrating accumulated deviation degrees.

### DETAILED DESCRIPTION

A technology has been known that predicts change in a human physical condition (mainly, deterioration of the physical condition) on the basis of external factors, such as weather conditions. For example, a service is conceivable that obtains weather conditions related to change in physical conditions, such as “the temperature difference in a day is 15 degrees or more” and “there are three consecutive days with a temperature in the morning equal to or less than ten degrees and a humidity equal to or less than 30%”, and issues a warning to the user if the weather condition satisfying a certain condition is predicted.

However, a technology of detecting change in the mental condition of the user has not been widely known.

An information processing apparatus according to this embodiment comprises a controller configured to execute: obtaining scheduled behavior information that is information about a scheduled behavior of a user; obtaining user behavior information that is information about a behavior performed by the user; calculating a deviation degree between the scheduled behavior and the behavior performed by the user, based on the user behavior information and the scheduled behavior information; and performing a predetermined process when the deviation degree exceeds a first value.

The scheduled behavior information is information about a scheduled behavior to be performed by the user. The scheduled behavior information is information about travel by the user, and may include, for example, a travel destination, a travel path, a departure time, and a required time period. The scheduled behavior information may be obtained based on a registered schedule of the user, or be estimated based on accumulated previous data.

The user behavior information is information about a behavior actually performed by the user. The information processing apparatus according to this embodiment obtains the deviation degree between a behavior scheduled to be performed by the user, and a behavior actually performed by the user, based on the scheduled behavior information and the user behavior information.

In case the mental condition deteriorates, a person sometimes cannot perform a scheduled behavior. Accordingly, by obtaining deviation degree between the scheduled behavior and the actually performed behavior, the mental condition of the user can be estimated. The controller performs a predetermined process on the basis of the deviation degree. Accordingly, for example, the user can be notified that there is a possibility that the mental condition deteriorates, and care can be taken for the user.

The user behavior information may include position information periodically obtained from a terminal carried by the user. The controller may calculate the deviation degree, based on the position information.

By periodic obtainment, and use of the accumulated position information on the user, the deviation degree between behaviors can be obtained.

The controller may determine a place that the user has dropped by, and may correct the deviation degree, based on the place that the user has dropped by.

In case the mental condition deteriorates, the dropping-by place during travel sometimes becomes unstable. Accordingly, based on the place that the user has dropped by, the calculated deviation degree may be corrected.

The controller may evaluate dropping-by appropriateness, for each place that the user has dropped by, and correct the deviation degree, based on a result of the evaluation.

It is conceivable that the user drops by during travel in an unscheduled manner. Accordingly, it may be individually evaluated whether dropping by is appropriate or not, and the deviation degree may be corrected based on the result. It can be evaluated whether dropping by is appropriate or not, based on the type and characteristics of the dropping-by place, and the dropping-by time slot, for example.

The scheduled behavior information may include a scheduled path that is a scheduled travel path of the user, and the controller may calculate a geographical deviation degree between the scheduled path and a path taken by the user.

The path taken by the user can be determined based on the periodically obtained position information, for example. As the geographical deviation degree between paths is higher, it can be determined that the user performs a behavior deviating from the scheduled one.

The scheduled behavior information may include time information related to a time period required for travel, and the controller may calculate a temporal deviation degree between the scheduled behavior and a behavior performed by the user.

The time information may be, for example, a scheduled passing time at a point on the path, a scheduled required time period in an interval included in the path, or the like. According to such a configuration, for example, it can be determined that the user is traveling at a significantly low pace.

The controller may calculate a comprehensive deviation degree, based on both the geographical deviation degree and the temporal deviation degree.

The two references that are “the deviation degree from the scheduled path” and “the deviation degree from the scheduled pace” are used together, which can accurately determine whether an abnormality is found in a behavior of the user.

The controller may be configured not to perform the predetermined process when the deviation degree exceeds a second value that is larger than the first value.

If the deviation degree is too high, there is a possibility that the scheduled behavior information (for example, an estimated behavior) is incorrect in the first place. Consequently, such a case may be excluded from the processing targets.

The controller may obtain data about a schedule of the user, and estimate the scheduled behavior of the user, based on the schedule.

The schedule of the user may be obtained with reference to a scheduler residing in the user terminal or a cloud, for example. The estimation may be made based on other information (for example, details of messages and emails transmitted and received by the user).

The controller may obtain a history of position information corresponding to the user, and estimate the scheduled behavior of the user, based on the history of the position information.

By using the history of the collected position information, a behavior pattern can be estimated. The controller can estimate the behavior of the user, on the basis of the previous history having similar conditions (the time slot, date, day of the week, weather, etc.), for example.

The controller may record the deviation degree, and perform the predetermined process when the deviation degree in a predetermined period exceeds a predetermined value.

By referring to the deviation degree in the predetermined period, it can be determined whether the state regarded to be abnormal continues or not. If the state regarded to be abnormal continues, the predetermined process is performed, which can more correctly take care.

The predetermined process may be a process of transmitting information related to mental health to an apparatus associated with the user.

The apparatus associated with the user may be a mobile terminal that the user carries, or an apparatus accessible by another user (e.g., a manager at a workplace, a family member, etc.) having a predetermined relationship with the user.

The predetermined process may be a process of transmitting information related to mental health to a server apparatus that manages the mobile body used by the user.

Accordingly, for example, to allow the user to receive care, measures, such as providing travel to a medical institution, and dispatching medical experts to the user, can be taken.

Hereinafter, referring to the drawings, embodiments of the present disclosure are described. Configurations in the following embodiments are only examples. The present disclosure is not limited to the configurations of the embodiments.

### First Embodiment

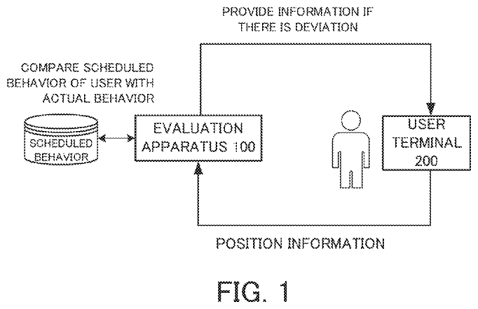

An overview of an evaluation system according to a first embodiment is described with reference to FIG. 1. The evaluation system according to this embodiment includes a user terminal 200 that is a terminal carried by a user, and an evaluation apparatus 100 that evaluates a behavior of the user.

The evaluation apparatus 100 is an apparatus that evaluates the behavior of the user, and provides the user with information when an abnormality is found in the behavior of the user. The evaluation apparatus 100 is configured to be capable of obtaining data about a scheduled behavior of the user, and determines whether the scheduled behavior coincides with the behavior that the user has actually performed, on the basis of position information obtained from the user terminal 200. When both deviate from each other, it is determined that the user becomes mentally unstable, and information is provided.

Note that in the example in FIG. 1, the evaluation apparatus 100 corresponds to the user terminal 200 on a one-to-one basis. Alternatively, a single evaluation apparatus 100 may support multiple user terminals 200.

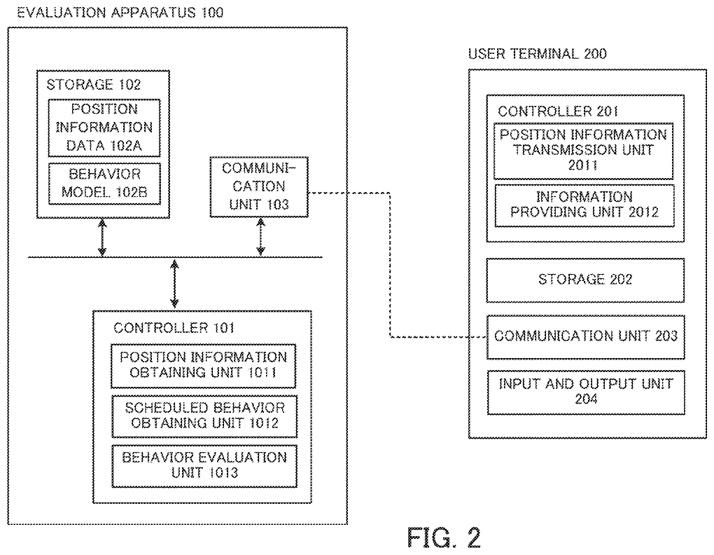

FIG. 2 is a diagram illustrating configuration elements of the evaluation system according to this embodiment in detail. Here, first, the user terminal 200 is described.

The user terminal 200 is a small-sized computer that is, for example, a smartphone, a mobile phone, a tablet computer, a personal information terminal, a notebook computer, or a wearable computer (smart watch or the like). The user terminal 200 includes a controller 201, a storage 202, a communication unit 203, and an input and output unit 204.

The controller 201 is an operation device that achieves control performed by the user terminal 200. The controller 201 can be achieved by an operation processing device, such as a CPU (Central Processing Unit).

The controller 201 includes two types of function modules that are a position information transmission unit 2011, and an information providing unit 2012. Each function module may be achieved by a CPU executing a program stored in the storage 202 described later.

The position information transmission unit 2011 obtains position information on the user terminal 200, and periodically transmits the information to the evaluation apparatus 100. The position information can be generated on the basis of a positioning results by the GPS, for example. The position information transmission unit 2011 may include a module that receives positioning signals transmitted from satellites, and outputs position information on the terminal.

The information providing unit 2012 provides the user with information through the input and output unit 204 described later, on the basis of data obtained from the evaluation apparatus 100.

The storage 202 includes a main memory and an auxiliary memory. The main memory is a memory where a program to be executed by the controller 201, and data used by the control program are deployed. The auxiliary memory is a device that stores a program to be executed by the controller 201, and data used by the control program. The auxiliary memory may store programs that are to be executed by the controller 201 and are packaged as applications. An operating system for executing the applications may be stored. The programs stored in the auxiliary memory are loaded on the main memory, and are executed by the controller 201, thereby processes described below are performed.

The main memory may include a RAM (Random Access Memory) and a ROM (Read Only Memory). The auxiliary memory may include an EPROM (Erasable Programmable ROM) and a hard disk drive (HDD). Furthermore, the auxiliary memory may include a removable medium, i.e., a removable recording medium. The removable medium is, for example, a USB (Universal Serial Bus) memory, or a disk recording medium, such as a CD (Compact Disc) or a DVD (Digital Versatile Disc).

The communication unit 203 is a wireless communication interface for connecting the user terminal 200 to a network. The communication unit 203 is configured to be communicable with the evaluation apparatus 100 through a wireless LAN or a mobile communication service, such as of 3G, LTE or 5G, for example.

The input and output unit 204 is a unit that accepts an input operation performed by the user, and presents information to the user. In this embodiment, the unit may be a single touch panel display. That is, the unit includes a liquid crystal display and control unit therefor, and a touch panel and control unit therefor.

Note that the configuration illustrated in FIG. 2 is one example. All or some of illustrated functions may be executed using a circuit designed in a dedicated manner. Alternatively, the program may be stored and executed by a combination of a main memory and an auxiliary memory that are not illustrated.

Next, the evaluation apparatus 100 is described.

The evaluation apparatus 100 is a server apparatus that executes a process of collecting position information from the user terminal 200 and generating a behavior model of the user, and a process of evaluating a behavior of the user by using the generated behavior model and providing information for the user on the basis of an evaluation result.

The evaluation apparatus 100 may be made up of a general-purpose computer. That is, the evaluation apparatus 100 may be made up of a computer that includes processors such as a CPU and a GPU, a main memory such as a RAM and a ROM, and an auxiliary memory such as an EPROM, a hard disk drive and a removable medium. The removable medium may be, for example, a USB memory, or a disk recording medium, such as a CD or a DVD. The auxiliary memory stores an operating system (OS), various programs, various tables and the like, loads the programs stored therein to a working area of the main memory and executes the programs, and controls each component through the execution of the program, which can achieve each function conforming with a predetermined object, as described later. Alternatively, some or all the functions may be achieved by a hardware circuit, such as an ASIC or FPGA.

A controller 101 is an operation device that achieves control performed by the evaluation apparatus 100. The controller 101 can be achieved by an operation processing device, such as a CPU.

The controller 101 includes three function modules that are a position information obtaining unit 1011, a scheduled behavior obtaining unit 1012, and a behavior evaluation unit 1013. Each function module may be achieved by the CPU executing the stored program.

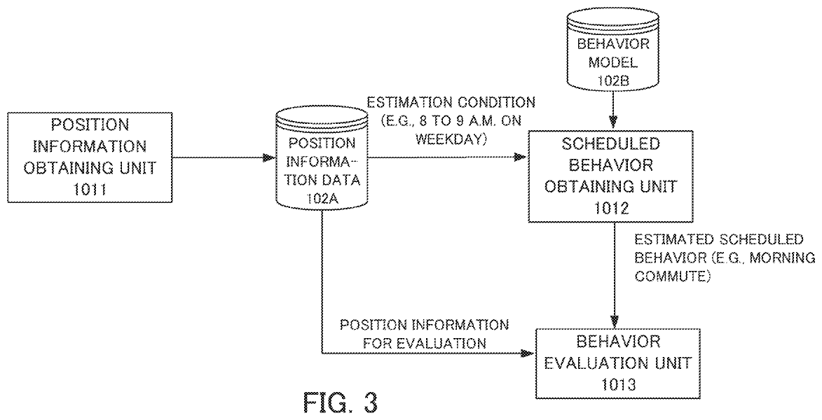

The three function modules are described with reference to FIG. 3 that is a diagram illustrating data transmitted and received between the modules.

The position information obtaining unit 1011 periodically obtains position information transmitted from the user terminal 200. The obtained data is associated with an identifier of the user, and is accumulated, as position information data 102A, in a storage 102 described later.

The scheduled behavior obtaining unit 1012 uses a model (behavior model 102B) for obtaining a behavior scheduled to be performed by the user (hereinafter, the scheduled behavior), thus obtaining the scheduled behavior of the user.

The behavior model 102B is a machine learning model obtained through learning of a typical behavior pattern to be performed by the user. The behavior model 102B is preliminarily constructed on the basis of position information data for learning (for example, a set of position information data items corresponding to a previous predetermined period).

By providing the behavior model with conditions (e.g., the date, time slot, day of the week, weather, etc.) serving as preconditions for estimation, an estimated scheduled behavior can be obtained.

The behavior evaluation unit 1013: (1) evaluates the deviation degree between the scheduled behavior of the user obtained by estimation and an actual behavior performed by the user on the basis of the position information data 102A obtained from the user terminal 200; and (2) provides information for the user on the basis of the evaluation result. The deviation degree between the behaviors can be evaluated in a geographical view and a temporal view. A specific method is described later.

The storage 102 includes a main memory and an auxiliary memory. The main memory is a memory where a program to be executed by the controller 101, and data used by the control program are deployed. The auxiliary memory is a device that stores a program to be executed by the controller 101, and data used by the control program.

The storage 102 stores the position information data 102A, and the behavior model 102B.

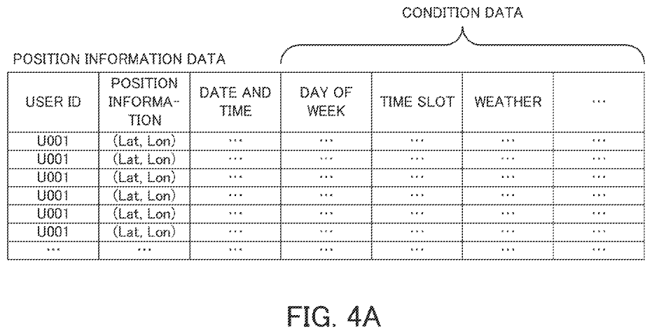

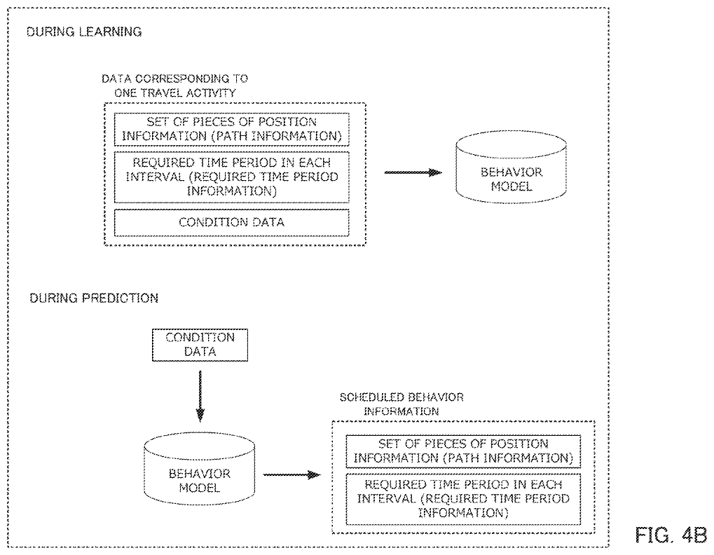

FIG. 4A illustrates an example of the position information data 102A. In this embodiment, items that include the position information (e.g., the latitude and longitude) on the user terminal 200, and the date and time are associated with the identifier of the user (user ID), and stored.

Among them, items usable as preconditions for estimating the behavior are called condition data.

For example, in a case where the behavior pattern of the user is changeable by the day of the week, time slot, weather and the like, these items may be stored as the condition data.

FIG. 4B is a diagram illustrating the behavior model 102B. The behavior model is a machine learning model obtained through learning of behaviors to be performed by the user under a predetermined condition (specifically, which path is taken for travel and which time period is required).

The behavior model 102B is constructed by execution of learning of data corresponding to travel occurring in the previous predetermined period, as training data.

The training data is a set of data items corresponding to one travel activity (for example, a travel activity from home to a workplace), and includes, for example, a set of pieces of position information, the required time period in each interval, and condition data. Note that the required time period in each interval can be obtained on the basis of date and time information included in the position information data.

Accordingly, by inputting the condition data, a model of outputting a set of pieces of position information (path information), and the required time period for each interval (required time period information), can be constructed. For example, under a condition “eight to nine a.m. on a weekday, weather: fine”, a path on which the user travels, and the required time period in each interval included in the path can be obtained.

Note that the behavior model 102B may be generated and stored on a user-by-user basis.

The data output by the behavior model can also be called scheduled behavior information. The path information included in the scheduled behavior information can also be called a scheduled path. Data about the position information collected from the user terminal 200 can also be called user behavior information.

The communication unit 103 is a communication interface similar to the communication unit 203. The communication unit 103 is configured to be communicable with the user terminal 200 via a wide area network, such as the Internet, for example.

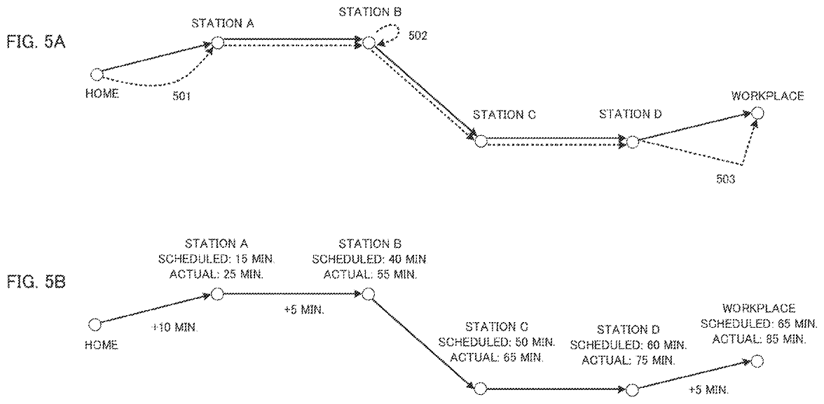

Next, the deviation between behaviors is described with reference to FIGS. 5A and 5B.

Solid lines indicate a path corresponding to the scheduled behavior of the user. FIG. 5A illustrates a path corresponding to a behavior “going to work”. This example describes that the user having left their home, passes through stations A, B, C and D and heads to the workplace while changing trains (symbols at the stations B and C indicate changing trains). Note that what is obtained by the evaluation apparatus 100 from the user terminal 200 is only the position information. However, the illustrated example clearly indicates the transit points (stations or the like) for the sake of convenience.

Here, it is assumed that the actually observed path of the user is what is indicated by broken lines.

For example, reference sign 501 indicates that the user having left home does not directly head to the station A. Reference sign 502 indicates that trains are not immediately changed, but a detour is made at the station B. Likewise, reference sign 503 indicates that the user having left the station D heads to the workplace while making a detour.

As described above, in case there are many deviations from the normally taken path, it can be estimated that the user is mentally unstable.

FIG. 5B is a diagram that associates the required time periods (elapsed time periods from departure) with a similar path. In the illustrated example, the travel time period is longer by 5 to 10 minutes than that in a normal case, in multiple intervals included in the path. As described above, if there is an interval requiring a longer time period for travel than that in the normal case, it can be estimated that the user is mentally unstable.

In this embodiment, the behavior evaluation unit 1013 compares the path corresponding to the scheduled behavior and the required time period with the actually observed path and the required time period, and calculates the deviation degree.

The deviation degree can be calculated by a method as described below, for example.

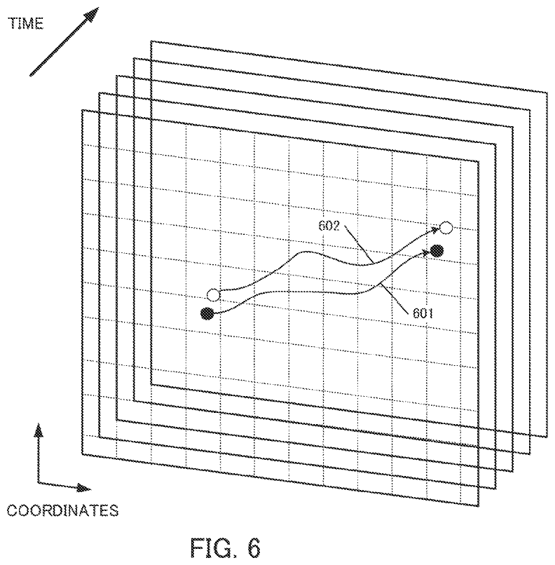

For example, as illustrated in FIG. 6, a set of points (reference sign 601) corresponding to the scheduled behavior, and a set of points (reference sign 602) corresponding to the actual behavior are arranged on a space having the coordinates on the X and Y axes and the time on the Z axis, and a value representing the distance between both is calculated. The calculated value is a value representing the deviation degree between the two behaviors. Here, when the calculated deviation degree exceeds a predetermined value, it can be determined that an unreasonable behavior deviating from a normal behavior pattern is performed.

Note that here, the distance in a time and space is calculated. Alternatively, the comparison target may only be the path. For example, only the geographical deviation degree between paths may be calculated. The comparison target may only be the time. For example, only the deviation degree between required time periods in respective intervals may be calculated.

The geographical deviation degree and the temporal deviation degree may be individually calculated, and these may be integrated and adopted as the final deviation degree.

Note that people do not necessarily behave in conformity with normal behavior patterns. For example, when they come home from workplaces, they drop by stores where they do not normally drop by, in some cases. However, in such cases, determination that an unreasonable behavior is performed is inappropriate. Accordingly, in this embodiment, erroneous determination is reduced by performing the following two processes.

(1) When the deviation degree is too large, it is construed to be a reasonable behavior.

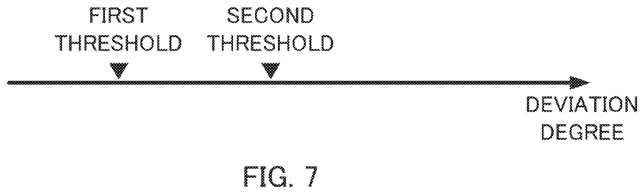

When the deviation degree from the scheduled behavior is large, it can be estimated that the user performs an unreasonable behavior. On the other hand, when the deviation degree from the scheduled behavior is far larger (for example, in a case where the final destination itself is different), it can be estimated that the user performs another behavior different from the scheduled behavior in the first place. Accordingly, two thresholds are set for the deviation degree. When the deviation degree is too large, it is construed to be a reasonable behavior. FIG. 7 is a diagram illustrating the relationship between the deviation degree and the threshold. As illustrated, when the deviation degree exceeds a second threshold, it may be determined to be another behavior different from the scheduled behavior, and regarded to be out of evaluation.

(2) The deviation degree is accumulated, and information is provided only when the threshold is exceeded.

If the difference is temporary even when the actual behavior is different from the scheduled behavior, it is not necessarily regarded to be problematic. For example, the daily behavior pattern may be evaluated and the deviation degree may be accumulated, and then only if the sum of the deviation degrees accumulated in a predetermined period exceeds a threshold, information may be provided.

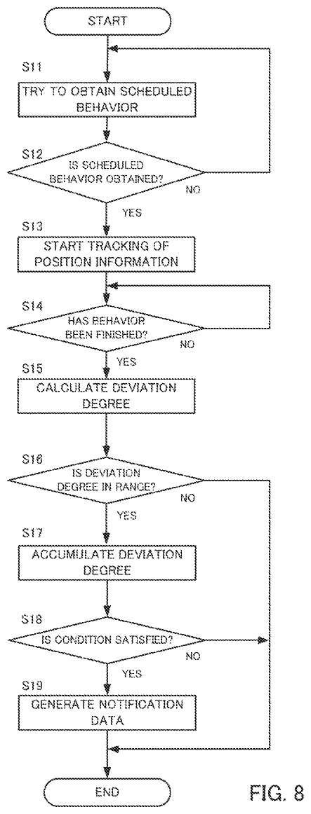

Next, the process performed by the controller 101 is described. FIG. 8 is a flowchart of an evaluation process performed by the controller 101.

It is assumed that a process of receiving and accumulating the position information from the user terminal 200 is periodically executed by the position information obtaining unit 1011 in a manner different from the illustrated process. It is assumed that the behavior model 102B corresponding to the target user is preliminarily constructed.

First, in step S11, the scheduled behavior obtaining unit 1012 tries to obtain the scheduled behavior using the behavior model 102B. Specifically, the condition data serving as preconditions (for example, the current date, time slot, day of the week, weather, etc.) is input into the behavior model 102B, and it is tried whether the scheduled behavior (path information and required time period information) is obtained or not.

If the scheduled behavior is obtained (step S12—Yes), the processing transitions to step S13, and the behavior evaluation unit 1013 starts to track the position information. If the scheduled behavior is not obtained (step S12—No), the processing returns to step S11.

In step S14, the behavior evaluation unit 1013 determines whether the user has reached the destination indicated by the scheduled behavior. If the user has reached the destination, the processing transitions to step S15. If the user has not reached the destination yet, tracking of the position information is continued. Even if the user has not reached the destination even after a lapse of a predetermined time period from departure, the processing may be terminated.

In step S15, the behavior evaluation unit 1013 calculates the deviation degree between the scheduled behavior and the actual behavior, on the basis of the set of pieces of position information and the required time period information obtained by tracking, and the path information and the required time period information included in the scheduled behavior.

In step S16, the behavior evaluation unit 1013 determines whether the calculated deviation degree is in a predetermined range or not. Specifically, it is determined whether or not the deviation degree is equal to or higher than a first threshold and less than a second threshold. If the condition is satisfied, the processing transitions to step S17. If the condition is not satisfied, the processing is finished.

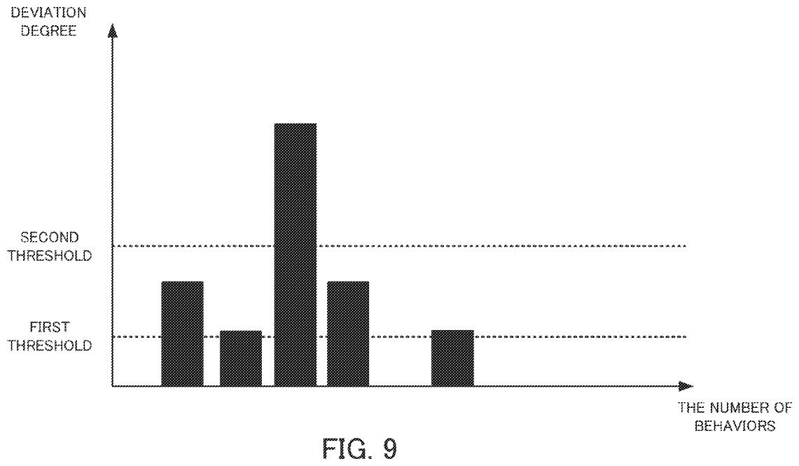

In step S17, the behavior evaluation unit 1013 accumulates the calculated deviation degree on a behavior-by-behavior basis. FIG. 9 illustrates an example of the accumulated deviation degrees.

In step S18, the behavior evaluation unit 1013 determines whether the accumulated deviation degree satisfies a predetermined condition or not. Specifically, the sum of deviation degrees in the previous predetermined period is calculated, and it is determined that the condition is satisfied if the calculated value exceeds a predetermined value.

If it is determined to be affirmative in step S18, the processing transitions to step S19, in which the behavior evaluation unit 1013 generates notification data. The notification data is information related to mental health, and includes, for example, a notification that the user requires care, and information about medical care. The notification data may be transmitted to the user terminal 200, or transmitted to a terminal or the like carried by another user (for example, a person managing and supervising the target user; typically, a guardian, a manager at a workplace, etc., hereinafter a supervisor) associated with the user.

When the notification data is transmitted to the user terminal 200, information is provided through the information providing unit 2012.

As described above, the evaluation apparatus according to the first embodiment detects that the user performs a behavior deviating from the previous behavior pattern, and generates the notification data. According to such a configuration, an abnormal behavior of the user having not been detectable can be detected, and early care can be taken.

### Modified Example of First Embodiment

In the first embodiment, the machine learning model for estimating the scheduled behavior of the user is used. Alternatively, the scheduled behavior of the target user may be what has been preliminarily generated.

For example, the supervisor of the target user may preliminarily create the scheduled behavior of the user, and store the scheduled behavior in the storage 102. In this case, the scheduled behavior obtaining unit 1012 obtains the scheduled behavior by referring not to the behavior model but to the preliminarily created data.

According to such a configuration, the supervisor can confirm whether a behavior of a person to be supervised deviates from the predetermined pattern or not (for example, commuting every day or not).

Furthermore, the scheduled behavior of the user may be generated on the basis of the schedule of the user. For example, the scheduled behavior obtaining unit 1012 may generate the scheduled behavior of the user on the basis of schedule information stored in the user terminal 200 or a cloud server. Furthermore, the behavior scheduled for the user may be determined on the basis of the details of email or a message transmitted and received by the user. Furthermore, the behavior model corresponding to the user may be stored outside of the evaluation apparatus 100 (e.g., a server apparatus).

### Second Embodiment

In the first embodiment, the deviation degree between behaviors is calculated based on the two references that are the geographical deviation degree and the temporal deviation degree. On the other hand, a second embodiment is an embodiment that individually evaluates places where the user has dropped by on the path, and further corrects the calculated deviation degree using the result.

For example, in the example in FIG. 5A, in a case where a dropping-by place with reference sign 503 is a place where many people drop by, such as a convenience store, it is inappropriate to evaluate that an abnormal behavior occurs. In contrast, when the user drops by a place with low appropriateness, for example, dropping by a park late at night, an abnormality is predicted.

In the second embodiment, the storage 102 includes data for evaluating dropping-by appropriateness. The behavior evaluation unit 1013 identifies a place that the user has dropped by, and evaluates the dropping-by appropriateness on the basis of the place and the dropping-by time slot. The deviation degree is corrected on the basis of the evaluation result of the appropriateness.

The place that the user has dropped by (hereinafter, the dropping-by place) can be determined by referring to data that is an aggregation of pieces of position information on estimated dropping-by places (latitudes and longitudes), for example.

The dropping-by appropriateness can be evaluated using the machine learning model, for example. For example, by using the machine learning model obtained by learning of the identifiers of dropping-by places, times and the number of visitors, as training data, it can be approximately estimated how many visitors are at any place and date and time. The larger the number of visitors is, the more appropriate the dropping by is estimated to be.

Note that the dropping-by appropriateness may be evaluated using what is other than the machine learning model. For example, data (hereinafter, appropriateness data) that associates the type, characteristics and time slot of the dropping-by place with a value indicating the user's dropping-by possibility may be stored in the storage 102, and be utilized.

In the second embodiment, for example, if the user has dropped by an eating place in a time slot for a meal, it can be evaluated that the appropriateness is high. In this case, the behavior evaluation unit 1013 corrects the calculated deviation degree such that the deviation degree can be smaller.

In contrast, if the user stays at a place with a low possibility of use during travel (e.g., a park, a riverside, etc.), it can be evaluated that the appropriateness is low. In this case, the behavior evaluation unit 1013 corrects the calculated deviation degree such that the deviation degree can be larger.

According to the second embodiment, it can be more correctly determined whether the user performs a reasonable behavior or not.

Note that in the second embodiment, based on the dropping-by place of the user, the deviation degree calculated in the first embodiment is corrected. Alternatively, the deviation degree between behaviors may be evaluated using only the dropping-by place.

For example, the evaluation apparatus 100 may refer to the appropriateness data described above. If the user has visited a place indicated by the appropriateness data, deviation from the normal behavior may be determined. In this case, the lower the value indicating the user's dropping-by possibility is, the higher the deviation degree can be achieved.

### Modified Example

The embodiments described above are only examples. The present disclosure can be implemented while being appropriately changed in a range without departing from the gist.

For example, processes and units described in the present disclosure can be freely combined and implemented only if no technical contradiction occurs.

For example, in the description of the embodiments, the evaluation apparatus 100 only provides information. A process for taking care for the user may be automatically executed. For example, a process of transmitting notification data to the user's doctor in charge and making a medical reservation may be executed. Furthermore, in a case where the user routinely uses a mobile body (e.g., an autonomous driven vehicle), the evaluation apparatus 100 may take an action for an apparatus that manages the mobile body. Accordingly, for example, the user can be recommended to access a medical institution, and a medical expert can be picked up and dropped off at the user.

In the description of the embodiments, the position information on the user is obtained from the user terminal 200. Alternatively, a unit for sensing the user may be provided, and position information may be generated on the basis of the sensing result.

In addition, the processing described as being performed by one device may be shared and executed by a plurality of devices. Alternatively, the processing described as being performed by different devices may be executed by one device. In a computer system, what hardware configuration (server configuration) realizes each function can be flexibly changed.

The present disclosure can also be realized by supplying a computer program including the functions described in the above embodiments to a computer and causing one or more processors included in the computer to read and execute the program. Such a computer program may be provided to the computer by a non-transitory computer-readable storage medium connectable to a system bus of the computer, or may be provided to the computer via a network. Examples of non-transitory computer readable storage media include: any type of disk such as a magnetic disk (floppy (registered trademark) disk, hard disk drive (HDD), etc.), an optical disk (CD-ROM, DVD disk, Blu-ray disk, etc.); and any type of medium suitable for storing electronic instructions, such as read-only memory (ROM), random access memory (RAM), EPROM, EEPROM, magnetic cards, flash memory, and optical cards.

What is claimed is:

1. An information processing apparatus, comprising a controller configured to execute:
obtaining scheduled behavior information that is information about a scheduled behavior of a user;
obtaining user behavior information that is information about a behavior performed by the user;
calculating a deviation degree between the scheduled behavior and the behavior performed by the user, based on the user behavior information and the scheduled behavior information; and
performing a predetermined process when the deviation degree exceeds a first value.

2. The information processing apparatus according to claim 1, wherein
the user behavior information includes position information periodically obtained from a terminal carried by the user, and
the controller calculates the deviation degree, based on the position information.

3. The information processing apparatus according to claim 2, wherein
the controller determines a place that the user has dropped by, and corrects the deviation degree, based on the place that the user has dropped by.

4. The information processing apparatus according to claim 3, wherein
the controller evaluates dropping-by appropriateness, for each place that the user has dropped by, and corrects the deviation degree, based on a result of the evaluation.

5. The information processing apparatus according to claim 2, wherein
the scheduled behavior information includes a scheduled path that is a scheduled travel path of the user, and
the controller calculates a geographical deviation degree between the scheduled path and a path taken by the user.

6. The information processing apparatus according to claim 5, wherein
the scheduled behavior information includes time information related to a time period required for travel, and
the controller calculates a temporal deviation degree between the scheduled behavior and a behavior performed by the user.

7. The information processing apparatus according to claim 6, wherein
the controller calculates a comprehensive deviation degree, based on both the geographical deviation degree and the temporal deviation degree.

8. The information processing apparatus according to claim 1, wherein
when the deviation degree exceeds a second value that is larger than the first value, the controller does not perform the predetermined process.

9. The information processing apparatus according to claim 1, wherein
the controller obtains data about a schedule of the user, and
estimates the scheduled behavior of the user, based on the schedule.

10. The information processing apparatus according to claim 1, wherein
the controller obtains a history of position information corresponding to the user, and
estimates the scheduled behavior of the user, based on the history of the position information.

11. The information processing apparatus according to claim 1, wherein
the controller records the deviation degree, and performs the predetermined process when the deviation degree in a predetermined period exceeds a predetermined value.

12. The information processing apparatus according to claim 1, wherein
the predetermined process is a process of transmitting information related to mental health to an apparatus associated with the user.

13. The information processing apparatus according to claim 1, wherein
the predetermined process is a process of transmitting information related to mental health to a server apparatus that manages a mobile body used by the user.

14. An information processing system, comprising: a user terminal carried by a user; and a server apparatus,
wherein the user terminal includes a first controller configured to periodically transmit position information to the server apparatus, and
the server apparatus includes a second controller configured to execute:
obtaining scheduled behavior information that is information about a scheduled behavior of the user;
generating user behavior information that is information about a behavior performed by the user, based on the position information transmitted from the user terminal;
calculating a deviation degree between the scheduled behavior and the behavior performed by the user, based on the user behavior information and the scheduled behavior information; and
performing a predetermined process when the deviation degree exceeds a first value.

15. The information processing system according to claim 14, wherein
the first controller periodically transmits the position information to the server apparatus, and
the second controller calculates the deviation degree, based on the position information.

16. The information processing system according to claim 15, wherein
the second controller determines a place that the user has dropped by, and corrects the deviation degree, based on the place that the user has dropped by.

17. The information processing system according to claim 15, wherein
the scheduled behavior information includes a scheduled path that is a scheduled travel path of the user, and
the second controller calculates a geographical deviation degree between the scheduled path and a path taken by the user.

18. The information processing system according to claim 17, wherein
the scheduled behavior information includes time information related to a time period required for travel, and
the second controller calculates a temporal deviation degree between the scheduled behavior and a behavior performed by the user.

19. The information processing system according to claim 14, wherein
the second controller estimates the scheduled behavior of the user, based on a history of the position information obtained from the user terminal.

20. A non-transitory computer readable storing medium recording a computer program for causing a computer to perform an information processing method comprising:
obtaining scheduled behavior information that is information about a scheduled behavior of a user;
obtaining user behavior information that is information about a behavior performed by the user;
calculating a deviation degree between the scheduled behavior and the behavior performed by the user, based on the user behavior information and the scheduled behavior information; and
performing a predetermined process when the deviation degree exceeds a first value.
